(19) United States
(12) Patent Application Publication
(10) Pub. No.: US 2015/0038980 A1
(43) Pub. Date: Feb. 5, 2015

(54) ROBOT FOR HOLDING AND FOR HANDLING MEDICAL INSTRUMENTS AND EQUIPMENT

(71) Applicants: Michael Merscher, Roedermark (DE); Ralf Gundling, Kronau (DE); Markus Schwarz, Ebenhausen (DE); Juergen Hesser, Heidelberg (DE); Peter P. Pott, Mannheim (DE); Robert Boesecke, Wiesloch (DE); Jens Brodersen, Ilvesheim (DE); Vitor Vieira, Heidelberg (DE); Eugen Lisiak, Mannheim (DE); Am Tuong Nguyen, Stuttgart (DE)

(72) Inventors: Michael Merscher, Roedermark (DE); Ralf Gundling, Kronau (DE); Markus Schwarz, Ebenhausen (DE); Juergen Hesser, Heidelberg (DE); Peter P. Pott, Mannheim (DE); Robert Boesecke, Wiesloch (DE); Jens Brodersen, Ilvesheim (DE); Vitor Vieira, Heidelberg (DE); Eugen Lisiak, Mannheim (DE); Am Tuong Nguyen, Stuttgart (DE)

(73) Assignee: Ruprecht-Karls-Universitaet Heidelberg, Heidelberg (DE)

(21) Appl. No.: 14/128,975

(22) PCT Filed: Apr. 23, 2012
(86) PCT No.: PCT/DE2012/200029

§ 371 Date: Apr. 18, 2014

(30) Foreign Application Priority Data
Jun. 24, 2011 (DE) 10 2011 105 748.3

(51) Int. Cl. A61B 19/00

(52) U.S. Cl. CPC A61B 19/2203; A61B 2019/464
USPC 606/130

(57) ABSTRACT

A robot for holding and for handling medical instruments/equipment (1), in particular retractors, preferably for use in orthopedic operations, comprises a manipulator (2) and an end effector (3) supported on the manipulator (2) for gripping/coupling of the particular instrument (1), wherein means for detecting external parameters relating to the holding situation are provided and wherein the holding/handling function of the robot can be defined on the basis of the identified parameters and optionally with the use of further predeterminable parameters.

### CROSS REFERENCE TO RELATED APPLICATIONS

This application is a national stage application, filed under 35 U.S.C. §371, of International Application No. PCT/DE2012/200029, filed Apr. 23, 2012, and German Application No. 10 2011 105 748.3, filed Jun. 24, 2011, all of which are hereby incorporated by reference in their entirety.

### BACKGROUND

1. Technical Field

The invention relates to a robot for holding and manipulating medical instruments/equipment, in particular retractors, preferably for use in orthopedic operations.

The holding and manipulating tasks in question may relate specifically to holding retractors during a surgical operation. Retractors are surgical holding instruments which are used to hold an operating area open.

2. Description of Related Art

Hitherto holding and manipulating tasks have generally been performed by medical staff. The retractors used for holding open the operating area must be held by the assistant in such a way that the surgeon has the best possible view and freedom of movement in order perform the operation. In this case it is particularly important that the tissue is not subjected to too much tension by the retractors in order avoid injuries to the patient. Therefore sometimes the retractors must be adjusted by the assistant if external disruptions or forces occur. Holding the retractors is an exhausting and time-consuming task which is physically strenuous and tiring. In particular in orthopedic operations, such as for example total hip replacement (THR) surgery, great forces occur, so that the task of holding the instrument is very strenuous for the assistant. Since the holding and manipulating tasks are very frequently performed by a qualified doctor, holding the retractors involves high costs.

Furthermore, devices are known in practice which are fixed for example on the operating table and on which a holding instrument or retractor can be mounted. To compensate for external disruptions, such as can occur in particular orthopedic operations, systems which are damped by compressed air are used. However, in this case it is problematic that the retractors are fixed rigidly on the holding frame, so that they are not actively adjustable. The leads to the possibility that the retractors loosen unnoticed or do not slacken when required, which can result inter alia in hindrances occurring in the course of the operation or even injuries to the patient's tissue and soft tissue. A further problem is that it is time-consuming and involves numerous staff to mount the rigid holding frame before the start of the actual operation, during which the holding frame must be kept sterile, but once again this incurs costs.

Finally, robots have been known for many years for use in surgical operations Simply by way of example reference is made in this connection to DE 102 39 673 A1, which shows a device for machining parts, in particular of bones, organs, etc. of the human and animal body. However, robot systems of this type are not suitable for holding and for manipulating medical instruments/equipment, such as for example retractors, since no adaptation of the holding process to external disruptions is provided. Rather, the robot systems in question operate by remote control, the robot reproducing the surgeon's hand movements.

### BRIEF SUMMARY

Therefore the object of the present invention is to design a robot for holding and for manipulating medical instruments, in particular retractors, in such a way that with minimal staffing costs it is possible to reliably perform exhausting and time-consuming holding and manipulating tasks which are sometimes subject to external disruptions.

This object is achieved according to the invention by the features of claim 1. According to this a robot for holding and for handling medical instruments/equipment is provided with a manipulator and an end effector supported by the manipulator for gripping/coupling of the particular instrument, wherein means for detecting external parameters relating to the holding situation are provided and wherein the holding/handling function of the robot can be defined on the basis of the identified parameters and optionally with the use of further predeterminable parameters.

In accordance with the invention it has been recognized that a robot can be used in an ideal manner for taking over the holding and manipulating tasks In this connection the robot has a manipulator and an end effector supported by the manipulator for gripping/coupling of the particular instrument. In accordance with the invention it has also been recognized that an active holding/handling function of the robot can be defined in a surprisingly simple manner if means for detecting external parameters relating to the holding situation are provided. This may for example relate to forces which occur, which can be detected by the robot and with the aid of which the holding/handling function of the robot can be defined. Finally it has been recognized that the holding/handling function of the robot using further predeterminable parameters, which for example the surgeon predetermines before or during the operation, can be further defined. The surgeon's knowledge and experience are therefore the basis for the holding/handling function of the robot. Thus these design features specify a robot which reliably performs the time-consuming and exhausting holding and manipulating tasks, so that for example retractors no longer have to be held by medical staff.

The means for detecting external parameters are advantageously designed as sensors for detecting external forces acting on the manipulator and/or on the end effector. The external forces can be converted into a corresponding movement of the manipulator and/or of the end effector, so that the end effector can be brought into a holding/handling position on the instrument. Due to this force-free guiding the robot or the end effector can be guided by the surgeon at a required point in space. In this case it is conceivable that by the surgeon or the medical staff the instrument or the retractor is already introduced into the operating area and is positioned according to the surgeon's requirements. At this point it may be noted that the surgeon thus predetermines in particular the force which the retractor exerts on the tissue. The robot can now be led to the instrument and can take over the already exactly positioned instrument from the surgeon. Due to this design feature, therefore, it is possible that the robot takes over a holding task which has been started by the surgeon and thus exactly defined. Furthermore it is conceivable that if need be, for example at the end of the operation, takes over the instrument again from the robot. Thus a holding task which the surgeon begins and also ends again would be taken over by the robot in the interim. Costly global tracking/positioning systems for guiding the robot to the instrument or the operating area are omitted.

With regard to an exact definition of the robot's holding/handling function, external parameters can be detected during or after the gripping/coupling of the instrument to the end effector. The forces occurring on the end effector and/or the orientation of the end effector in space and/or the working area limits of the end effector can be detected for example as external parameters. Due to this design feature a calibration of the robot with the instrument or with the retractor is achieved indirectly. During or after the gripping/coupling of the instrument on the end effector, for example, the sensors detect the increase in force until no further significant increase in force can be recorded. Furthermore, by recording of the orientation of the end effector in space it is conceivable to calibrate the robot relative to the instrument or the retractor. Thus a time-consuming calibration of the robot before surgical use is unnecessary.

In relation to an active regulation of the holding/handling function it is particularly advantageous if the necessary adjustment parameters for the holding/handling function can be determined from the external parameters and optionally from the predeterminable parameters for example by an adjustment of the force and/or the impedance or by a hybrid force and position adjustment.

An input device can be provided for input of the predeterminable parameters, such as for example the damping characteristics of the viscoelastic tissue. The input device may be designed as a keyboard or touch-screen. Furthermore it is conceivable that the input device for activating and deactivating the holding/handling function is preferably designed as a foot switch.

In order to ensure an active holding/handling function of the robot, the means for detecting external parameters may be designed as sensors for detecting the forces occurring between the instrument and the held tissue during the holding situation. Due to these design features, when a predetermined force value is exceeded the end effector can be reset automatically in the direction in which the force is occurring. For example before the start of the operation the surgeon may define a maximum force parameter via an input device, so that when this value is exceeded the end effector can be reset automatically in the direction in which the force is occurring. Furthermore is conceivable that an acoustic and/or optical signal is triggered when the measured value exceeds the predefined force value. The risk of hindrances in the conduct of the operation, which may be caused for example by rigid retaining frames, is considerably minimized, since the robot carries out an active holding/handling function on the instrument.

With regard to the simplest possible design which meets the special requirements of a medical operating device, the end effector is advantageously made up of a plurality of modules. The modules are preferably connected to one another by means of coupling elements. The modules can be releasably connected to one another in order to dismantle and to sterilize the individual components of the end effector in a short time.

For gripping or coupling the instrument on the end effector, the end effector may have a gripper module. The gripper module may be designed for example as an individual gripper. In order to adapt the robot to various standardized or also non-standardized medical instruments/equipment, the gripper module may be designed to be interchangeable. Depending upon the operation and the medical instrument required for this purpose, the gripper module may be selected and mounted appropriately on the end effector. For security against an unexpected release of the instruments/equipment from the gripper module, the gripper module may have an action system and/or a kinematic system. In a particularly advantageous manner the kinematic system is designed in such a way that it is independent of an external power supply. As a result the kinematic system can be opened or closed at any time by the assistant/surgeon. In particular in the event of failure of the robot—for example in the event of “freezing”—the surgeon can open the kinematic system and take over the holding instrument from the end effector. Because of the modular construction of the end effector the gripper module can then be removed, so that sufficient working space is available for the surgeon.

The sensors required for detection of the external parameters can be accommodated in a particularly advantageous manner in a sensor module of the end effector. The sensor module can be directly mounted on the robot flange of the manipulator, for example by means of a mechanical interface. Furthermore it is conceivable that the sensor module has sensors for detecting internal parameters, so that for example it is possible to detect which gripper module is mounted on the end effector, so that the corresponding parameters for controlling of the robot are loaded.

Since the robot must be used under sterile conditions, an isolation module for separating a sterile area from an unsterile area of the end effector can be provided in a particularly advantageous manner on the end effector. The isolation module can be disposed for example between the sensor module and the gripper module, so that the sensor module is located in the unsterile area of the end effector. In a particularly advantageous manner the unsterile area of the end effector is provided with a replaceable sterile covering, for example a film. In specific terms, the film can be releasably fixed on the isolation module and can extend beyond the sensor module over the manipulator. Thus the film can be replaced before every operation, so that the unsterile area of the end effector or manipulator is always covered.

In order to further minimize the preparation time a position detection system can be provided, so that on the basis of the detected position data the manipulator can be brought automatically into an optimal initial position for guiding into the holding/handling position. The force-free guiding of the manipulator or end effector on the instrument still has to take place for example in a straight line, so that the handover time is significantly reduced.

In a further advantageous manner it is possible to provide a plurality of manipulators with an end effector carried by the manipulator, so that several or all of the instruments/equipment required for keeping the operating area open can be manipulated by the robot.

### DETAILED DESCRIPTION OF THE DRAWINGS

There are now various possibilities for configuring and modifying the teaching of the present invention in an advantageous manner In this connection reference is made on the one hand to the claims following Claim 1 and on the other hand to the following explanation of a preferred embodiment of the invention with the aid of the drawing. In conjunction with the explanation of the preferred embodiment of the invention with reference to the drawings, preferred embodiments and modifications of the teaching are also explained in general. In the drawings:

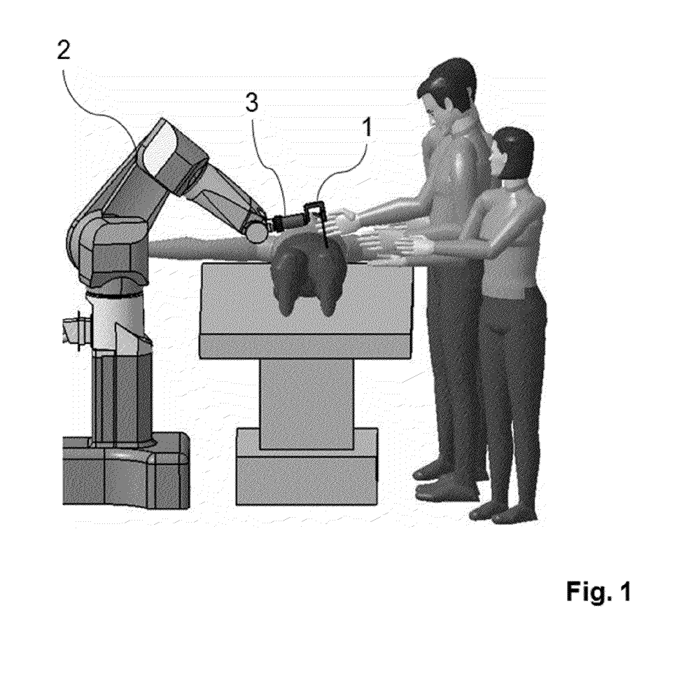

FIG. 1 shows a schematic representation of an embodiment of a robot according to the invention,

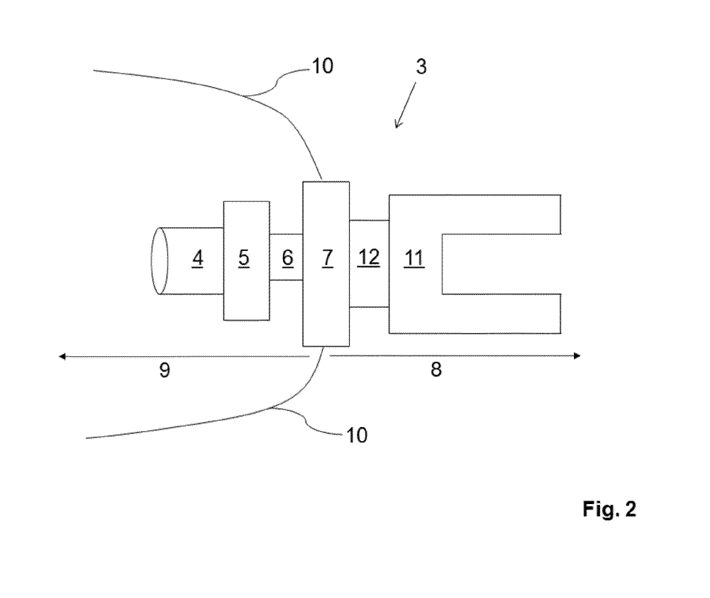

FIG. 2 shows a schematic representation of an embodiment of an end effector according to the invention, and

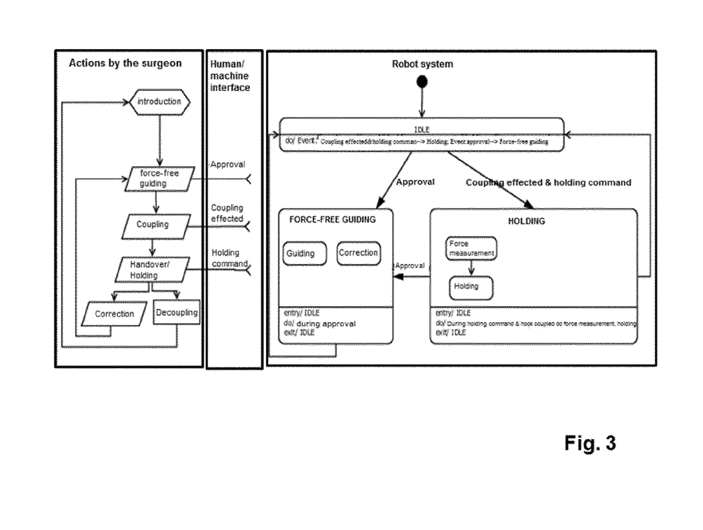

FIG. 3 shows a flow diagram for schematic representation of an embodiment of the system architecture of a robot according to the invention.

### DETAILED DESCRIPTION OF VARIOUS EMBODIMENTS

FIG. 1 shows a schematic representation of a robot according to the invention for holding a retractor 1 during a surgical operation. The robot has an end effector 3 supported by a manipulator 2. The end effector 3 serves for gripping and holding the retractor 1. Since the robot actively carries out the holding function independently, the robot can react to external disruptions and can for example reset the retractor. As a result the risk of disruptions of the conduct of the operation is reduced to a minimum.

FIG. 2 shows a schematic representation of an embodiment of an end effector 3 according to the invention. The end effector 3 is fixed on the robot flange 4 of the manipulator. For the sake of simplicity the manipulator is not shown in FIG. 2. The end effector 3 has a sensor module 5. Sensors for detecting internal and external parameters are provided in the sensor module 5. The isolation module 7 is fixed on the sensor module 5 via a first coupling element 6. The isolation module 7 serves for sub-division of the end effector 3 into a sterile area 8 and an unsterile area 9. For this purpose the isolation module 7 has a sterile cover 10 which is for example configured as a film. The area 9 below the sterile cover 10, i.e. the manipulator 2 together with the robot flange 4 and the sensor module 5, is regarded as unsterile.

In the sterile area 8 above the cover 10 the gripper module 11 is fixed on the isolation module 7 by a second coupling element 12. The gripper module 11 is configured here as an individual gripper and serves for gripping/coupling of the instrument. The sensors in the sensor module 5 detect as internal parameter which gripper module 11 is installed on the isolation module 7, in order to load the corresponding parameters for control of the robot. Since the isolation module 7, the second coupling element 12 and the gripper module 11 are located in the sterile area 8, they must be designed to be sterilisable.

FIG. 3 shows a flow diagram for schematic representation of an embodiment of the system architecture of a robot according to the invention. The states of the robot can be controlled by means of the surgeon's action via the human/machine interface. These states include approval for force-free guiding, attachment/coupling of a retractor or holding instrument as well as the command to carry out the holding function. The command to carry out the holding function is given via an input device, for example via a foot switch.

In accordance with the invention it has been recognized that a robot can be used in an ideal manner for taking over the holding and manipulating tasks In this connection the robot has a manipulator and an end effector supported by the manipulator for gripping/coupling of the particular instrument. In accordance with the invention it has also been recognized that an active holding/handling function of the robot can be defined in a surprisingly simple manner if means for detecting external parameters relating to the holding situation are provided. This may for example relate to forces which occur, which can be detected by the robot and with the aid of which the holding/handling function of the robot can be defined. Finally it has been recognized that the holding/handling function of the robot using further predeterminable parameters, which for example the surgeon predetermines before or during the operation, can be further defined. The surgeon's knowledge and experience are therefore the basis for the holding/handling function of the robot. Thus these design features specify a robot which reliably performs the time-consuming and exhausting holding and manipulating tasks, so that for example retractors no longer have to be held by medical staff.

The means for detecting external parameters are advantageously designed as sensors for detecting external forces acting on the manipulator and/or on the end effector. The external forces can be converted into a corresponding movement of the manipulator and/or of the end effector, so that the end effector can be brought into a holding/handling position on the instrument. Due to this force-free guiding the robot or the end effector can be guided by the surgeon at a required point in space. In this case it is conceivable that by the surgeon or the medical staff the instrument or the retractor is already introduced into the operating area and is positioned according to the surgeon's requirements. At this point it may be noted that the surgeon thus predetermines in particular the force which the retractor exerts on the tissue. The robot can now be led to the instrument and can take over the already exactly positioned instrument from the surgeon. Due to this design feature, therefore, it is possible that the robot takes over a holding task which has been started by the surgeon and thus exactly defined. Furthermore it is conceivable that if need be, for example at the end of the operation, takes over the instrument again from the robot. Thus a holding task which the surgeon begins and also ends again would be taken over by the robot in the interim. Costly global tracking/positioning systems for guiding the robot to the instrument or the operating area are omitted.

With regard to an exact definition of the robot's holding/handling function, external parameters can be detected during or after the gripping/coupling of the instrument to the end effector. The forces occurring on the end effector and/or the orientation of the end effector in space and/or the working area limits of the end effector can be detected for example as external parameters. Due to this design feature a calibration of the robot with the instrument or with the retractor is achieved indirectly. During or after the gripping/coupling of the instrument on the end effector, for example, the sensors detect the increase in force until no further significant increase in force can be recorded. Furthermore, by recording of the orientation of the end effector in space it is conceivable to calibrate the robot relative to the instrument or the retractor. Thus a time-consuming calibration of the robot before surgical use is unnecessary.

In relation to an active regulation of the holding/handling function it is particularly advantageous if the necessary adjustment parameters for the holding/handling function can be determined from the external parameters and optionally from the predeterminable parameters for example by an adjustment of the force and/or the impedance or by a hybrid force and position adjustment.

An input device can be provided for input of the predeterminable parameters, such as for example the damping characteristics of the viscoelastic tissue. The input device may be designed as a keyboard or touch-screen. Furthermore it is conceivable that the input device for activating and deactivating the holding/handling function is preferably designed as a foot switch.

In order to ensure an active holding/handling function of the robot, the means for detecting external parameters may be designed as sensors for detecting the forces occurring between the instrument and the held tissue during the holding situation. Due to these design features, when a predetermined force value is exceeded the end effector can be reset automatically in the direction in which the force is occurring. For example before the start of the operation the surgeon may define a maximum force parameter via an input device, so that when this value is exceeded the end effector can be reset automatically in the direction in which the force is occurring. Furthermore is conceivable that an acoustic and/or optical signal is triggered when the measured value exceeds the predefined force value. The risk of hindrances in the conduct of the operation, which may be caused for example by rigid retaining frames, is considerably minimized, since the robot carries out an active holding/handling function on the instrument.

With regard to the simplest possible design which meets the special requirements of a medical operating device, the end effector is advantageously made up of a plurality of modules. The modules are preferably connected to one another by means of coupling elements. The modules can be releasably connected to one another in order to dismantle and to sterilize the individual components of the end effector in a short time.

For gripping or coupling the instrument on the end effector, the end effector may have a gripper module. The gripper module may be designed for example as an individual gripper. In order to adapt the robot to various standardized or also non-standardized medical instruments/equipment, the gripper module may be designed to be interchangeable. Depending upon the operation and the medical instrument required for this purpose, the gripper module may be selected and mounted appropriately on the end effector. For security against an unexpected release of the instruments/equipment from the gripper module, the gripper module may have an action system and/or a kinematic system. In a particularly advantageous manner the kinematic system is designed in such a way that it is independent of an external power supply. As a result the kinematic system can be opened or closed at any time by the assistant/surgeon. In particular in the event of failure of the robot—for example in the event of “freezing”—the surgeon can open the kinematic system and take over the holding instrument from the end effector. Because of the modular construction of the end effector the gripper module can then be removed, so that sufficient working space is available for the surgeon.

The sensors required for detection of the external parameters can be accommodated in a particularly advantageous manner in a sensor module of the end effector. The sensor module can be directly mounted on the robot flange of the manipulator, for example by means of a mechanical interface. Furthermore it is conceivable that the sensor module has sensors for detecting internal parameters, so that for example it is possible to detect which gripper module is mounted on the end effector, so that the corresponding parameters for controlling of the robot are loaded.

Since the robot must be used under sterile conditions, an isolation module for separating a sterile area from an unsterile area of the end effector can be provided in a particularly advantageous manner on the end effector. The isolation module can be disposed for example between the sensor module and the gripper module, so that the sensor module is located in the unsterile area of the end effector. In a particularly advantageous manner the unsterile area of the end effector is provided with a replaceable sterile covering, for example a film. In specific terms, the film can be releasably fixed on the isolation module and can extend beyond the sensor module over the manipulator. Thus the film can be replaced before every operation, so that the unsterile area of the end effector or manipulator is always covered.

In order to further minimize the preparation time a position detection system can be provided, so that on the basis of the detected position data the manipulator can be brought automatically into an optimal initial position for guiding into the holding/handling position. The force-free guiding of the manipulator or end effector on the instrument still has to take place for example in a straight line, so that the handover time is significantly reduced.

In a further advantageous manner it is possible to provide a plurality of manipulators with an end effector carried by the manipulator, so that several or all of the instruments/equipment required for keeping the operating area open can be manipulated by the robot.

Finally it may be explicitly pointed out that the embodiments of the device according to the invention described above serve merely for explanation of the claimed teaching, but do not limit this to the embodiments.

### LIST OF REFERENCE SIGNS

1 retractor
2 manipulator
3 end effector
4 robot flange
5 sensor module
6 first coupling element
7 isolation module
8 sterile area
9 unsterile area
10 sterile cover
11 gripper module
12 second coupling element

1-17. (canceled)

18. Robot for holding and for handling medical instruments or equipment (1), such as retractors, for use in orthopedic operations, the robot comprising:
a manipulator (2); and
an end effector (3) supported on the manipulator (2) for at least one of gripping or coupling of the particular instrument (1),
wherein:
means for detecting external parameters relating to the holding situation are provided; and
the at least one of holding or handling functions of the robot are defined on the basis of the detected external parameters.

19. Robot according to claim 18, wherein the at least one of holding or handling functions of the robot are further defined on the basis of pre-determinable parameters, the pre-determinable parameters being separate and distinct from the detected external parameters.

20. Robot according to claim 18, wherein:
the means for detecting external parameters is configured as sensors for detecting external forces acting on at least one of the manipulator (2) or the end effector (3); and
the external forces can be converted into a corresponding movement of at least one of the manipulator (2) or the end effector (3), so that the end effector (3) can be brought into a holding/handling position on the instrument (1).

21. Robot according to claim 18, wherein at least one of during or after the gripping/coupling of the instrument (1) on the end effector (3), the forces occurring on the end effector (3) or the orientation of the end effector (3) in space or the working area limits of the end effector (3) can be detected as external parameters.

22. Robot according to claim 18, wherein the necessary adjustment parameters for the holding/handling function can be determined from the external parameters by an adjustment of at least one of the force or the impedance, or by a hybrid force and position adjustment.

23. Robot according to claim 19, wherein the necessary adjustment parameters for the holding/handling function can be determined from the pre-determinable parameters by an adjustment of at least one of the force or the impedance, or by a hybrid force and position adjustment.

24. Robot according to claim 18, wherein an input device is provided for activation and deactivation of the holding/handling function.

25. Robot according to claim 19, wherein an input device is provided for input of the predeterminable parameters.

26. Robot according to claim 18, wherein the means for detecting external parameters are designed as sensors for detecting the forces occurring between the instrument (1) and the held tissue during the holding situation, so that when a predetermined force value is exceeded the end effector (3) can be automatically reset in the direction of the force which is occurring.

27. Robot according to claim 18, wherein the end effector (3) is made up of several modules (5, 7, 11).

28. Robot according to claim 27, wherein the modules (5, 7, 11) are connected to one another, preferably releasably, via coupling elements (6, 12).

29. Robot according to claim 28, wherein the modules (5, 7, 11) are releasably connected to one another via the coupling elements (6, 12).

30. Robot according to claim 27, wherein the end effector (3) has a gripper module (11) preferably configured as an individual gripper.

31. Robot according to claim 30, wherein the gripper module (11) is designed to be replaceable.

32. Robot according to claim 30, wherein the gripper module (11) has at least one of an action system or a kinematic system.

33. Robot according to claim 32, wherein the kinematic system is independent of an external power supply, so that also in the event of a failure of the robot the medical instrument can be reliably removed from the gripper module.

34. Robot according to claim 27, wherein the end effector (3) has a sensor module (5), wherein the sensor module (5) comprises the sensors for detecting the external parameters.

35. Robot according to claim 27, wherein the end effector (3) has an isolation module (7) for separating a sterile area (8) from an unsterile area (9) of the end effector (3).

36. Robot according to claim 35, wherein the unsterile area (9) of the end effector (3) is covered with a sterile cover (10), preferably a film.

37. Robot according to claim 36, wherein the sterile cover (10) is a film.

38. Robot according to claim 18, wherein a position detection system is provided, so that on the basis of the detected position data the manipulator (2) can be brought automatically into an optimal initial position for guiding into the holding/handling position.

39. Device according to claim 18, wherein a plurality of manipulators (2) are provided.
